(19) United States
(12) Patent Application Publication
(10) Pub. No.: US 2015/0018679 A1
(43) Pub. Date: Jan. 15, 2015

(54) ULTRASONIC MEASURING DEVICE, ULTRASONIC IMAGE DEVICE, AND ULTRASONIC MEASURING METHOD

(71) Applicant: SEIKO EPSON CORPORATION, TOKYO (JP)

(72) Inventor: Kogo ENDO, Chino (JP)

(21) Appl. No.: 14/324,529

(22) Filed: Jul. 7, 2014

(30) Foreign Application Priority Data
Jul. 11, 2013 (JP) 2013-145225

(51) Int. Cl. A61B 8/08; A61B 5/00

(52) U.S. Cl. CPC A61B 8/485; A61B 5/418
USPC 600/438

(57) ABSTRACT

An ultrasonic measuring device including: an ultrasonic transducer device; a force sensor that measures pressing force; an emission unit that performs processing for emitting an ultrasonic beam; a reception unit that performs processing for receiving an ultrasonic echo obtained from the ultrasonic beam being reflected by a test subject; and a processing unit that performs analysis processing based on a reception signal from the reception unit and detection information from the force sensor, wherein the processing unit obtains elasticity information of a biological tissue layer of the test subject based on thickness information and pressing force information, the thickness information being thickness information of the biological tissue layer acquired based on the reception signal from the reception unit, and the pressing force information being pressing force information regarding the pressing force applied to the test subject from the force sensor.

### BACKGROUND

1. Technical Field

The present invention relates to an ultrasonic measuring device, an ultrasonic image device, an ultrasonic measuring method, and the like.

2. Related Art

Lymph node dissection has been performed as a treatment of breast cancer, but lymphedema, which may occur as a sequel after the dissection, is becoming a problem. As a method for detecting a lesion, such as lymphedema, that has an elastic constant different from that of normal biological tissue, detecting hardness (elastic constant) of biological tissue is known. For example, JP-A-10-118062 discloses a method in which an ultrasonic vibrator and a frequency measuring unit for detecting a resonance frequency of the ultrasonic vibrator are provided, and the hardness of an object is detected from the measured resonance frequency.

### SUMMARY

With this method, it is possible to see the relative difference with respect to the hardness of biological tissue, but this method is problematic in that it is difficult to obtain absolute values, and it is necessary to sacrifice reception sensitivity of the ultrasound image itself because the signal electrode of the ultrasonic vibrator is divided into two. An advantage of some aspects of the invention is to provide an ultrasonic measuring device, an ultrasonic image device, an ultrasonic measuring method, and the like that can measure elasticity information of biological tissue without affecting ultrasound reception sensitivity.

A first aspect of the invention relates to an ultrasonic measuring device including: an ultrasonic transducer device; a force sensor that measures pressing force; an emission unit that performs processing for emitting an ultrasonic beam; a reception unit that performs processing for receiving an ultrasonic echo obtained from the ultrasonic beam being reflected by a test subject; and a processing unit that performs analysis processing based on a reception signal from the reception unit and detection information from the force sensor, wherein the processing unit obtains elasticity information of a biological tissue layer of the test subject based on thickness information and pressing force information, the thickness information being thickness information of the biological tissue layer acquired based on the reception signal from the reception unit, and the pressing force information being pressing force information regarding the pressing force applied to the test subject from the force sensor.

According to this aspect of the invention, the processing unit can obtain the elasticity information of the biological tissue layer of the test subject through ultrasonic measurement, and it is therefore possible to detect, for example, a lesion, such as lymphedema, having an elastic constant different from that of normal biological tissue layers, with high precision. Furthermore, the force sensor that measures pressing force does not affect the emission and reception of ultrasound, and thus the user can measure the elastic constant while viewing the ultrasonic image such as a B mode image during measurement. As a result, it is possible to efficiently detect the lesion.

Also, in the first aspect of the invention, if the biological tissue layer includes a first biological tissue layer and a second biological tissue layer that have different elastic constants, the processing unit may acquire first pressing force information as the pressing force information, and first thickness information of the first biological tissue layer and first thickness information of the second biological tissue layer as the thickness information when the pressing force applied to the test subject during measurement is a first pressing force, acquire second pressing force information as the pressing force information, and second thickness information of the first biological tissue layer and second thickness information of the second biological tissue layer as the thickness information when the pressing force applied to the test subject during measurement is a second pressing force that is different from the first pressing force, and obtain elasticity information of the first biological tissue layer and elasticity information of the second biological tissue layer based on the first pressing force information, the second pressing force information, the first thickness information of the first biological tissue layer, the first thickness information of the second biological tissue layer, the second thickness information of the first biological tissue layer, and the second thickness information of the second biological tissue layer that have been acquired.

According to this configuration, the processing unit can separately obtain elasticity information of two biological tissue layers having different elastic constants. As a result, for example, a lesion such as lymphedema in a normal biological tissue layer can be detected with high precision separately from other portions. Furthermore, because the elasticity information of the lesion can be obtained with high precision separately from other portions, progression and the like of the lesion can be precisely recognized.

Also, in the first aspect of the invention, where the first pressing force is represented by F1, the second pressing force is represented by F2, the first thickness of the first biological tissue layer is represented by Ya1, the first thickness of the second biological tissue layer is represented by Yb1, the second thickness of the first biological tissue layer is represented by Ya2, the second thickness of the second biological tissue layer is represented by Yb2, initial thickness of the first biological tissue layer is represented by Ya0, initial thickness of the second biological tissue layer is represented by Yb0, the elasticity of the first biological tissue layer is represented by Ka, and the elasticity of the second biological tissue layer is represented by Kb, the processing unit may obtain the elasticity Ka of the first biological tissue layer and the elasticity Kb of the second biological tissue layer from the following relational equations: F1=Ka×(Ya1−Ya0)+Kb×(Yb1−Yb0); and F2=Ka×(Ya2−Ya0)+Kb×(Yb2−Yb0).

According to this configuration, the processing unit can obtain the elasticity information of each of the two biological tissue layers by solving the simultaneous equations.

Also, in the first aspect of the invention, the processing unit may generate ultrasonic image data based on a reception signal from the reception unit, analyze the ultrasonic image data so as to acquire thickness information of the biological tissue layer, and obtain the elasticity information of the biological tissue layer based on the acquired thickness information of the biological tissue layer and the pressing force information.

According to this configuration, the processing unit can acquire accurate thickness information of the biological tissue layer through analysis processing of the ultrasonic image data, and it is therefore possible to obtain more highly precise elasticity information.

Also, in the first aspect of the invention, the processing unit may detect a boundary in the biological tissue layer by detecting luminance value peaks in the ultrasonic image data, and obtain the thickness information of the biological tissue layer from coordinate values in a depth direction of the detected boundaries in the biological tissue layer.

According to this configuration, the processing unit can acquire thickness information of the biological tissue layer based on the ultrasonic image data.

Also, in the first aspect of the invention, the processing unit may extract a feature point of the biological tissue layer in the ultrasonic image data, and detects the boundary in the biological tissue layer based on displacement amount information of the feature point by application of the pressing force on the test subject.

According to this configuration, the processing unit can detect the boundary between biological tissue layers with higher accuracy by extracting feature points of the biological tissue layers in the ultrasonic image data.

Another aspect of the invention relates to an ultrasonic image device including: any one of the ultrasonic measuring devices described above; and a display unit that displays display image data generated by the processing unit.

Another aspect of the invention relates to an ultrasonic measuring method including: performing processing for emitting an ultrasonic beam to a test subject; performing processing for receiving an ultrasonic echo obtained from the ultrasonic beam being reflected by the test subject; acquiring pressing force information regarding pressing force applied to the test subject during measurement; and obtaining elasticity information of a biological tissue layer of the test subject based on the pressing force information and thickness information of the biological tissue layer based on a reception signal of the ultrasonic echo.

### BRIEF DESCRIPTION OF THE DRAWINGS

The invention will be described with reference to the accompanying drawings, wherein like numbers reference like elements.

FIG. 1 shows an example of the basic configurations of an ultrasonic measuring device and an ultrasonic image device.

FIGS. 2A and 2B are diagrams illustrating measurement of elasticity information performed by the ultrasonic measuring device.

FIG. 3 shows an example of a flowchart of elasticity information measurement.

FIG. 4 shows an example of a flowchart of processing for acquiring the thicknesses of biological tissue layers through feature point extraction performed on ultrasonic images.

FIGS. 5A and 5B show an example of feature point extraction.

FIGS. 6A and 6B show an example of feature point matching.

FIGS. 7A and 7B show an example of coordinate values of feature points.

FIGS. 8A, 8B and 8C show a configuration example of an ultrasonic transducer element.

FIG. 9 shows a configuration example of an ultrasonic transducer device.

FIGS. 10A and 10B show examples of ultrasonic transducer element groups.

FIGS. 11A and 11B show specific configuration examples of ultrasonic image devices.

### DESCRIPTION OF EXEMPLARY EMBODIMENTS

The following is a detailed description of preferred embodiments of the invention. Note that the embodiments described below are not intended to unduly limit the content of the invention recited in the claims, and all of the configurations described in the embodiments are not necessarily essential as solutions provided by the invention.

1. Basic Configuration Example

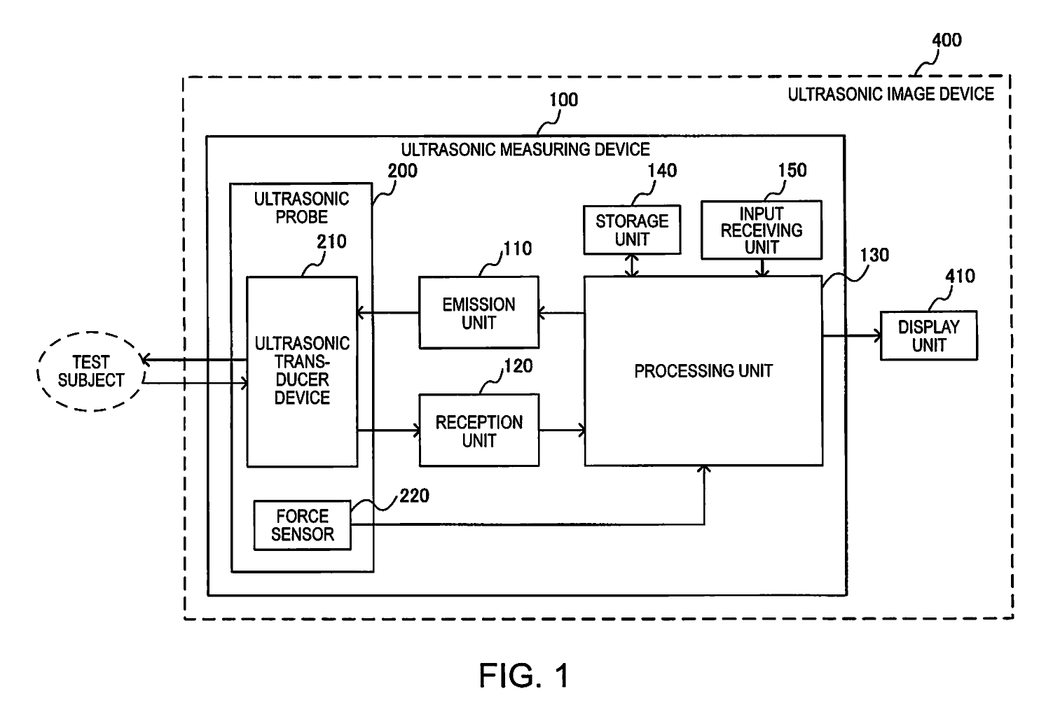

FIG. 1 shows an example of the basic configurations of an ultrasonic measuring device 100 and an ultrasonic image device 400 of this embodiment. The ultrasonic measuring device 100 of this embodiment includes an ultrasonic probe 200, an emission unit 110, a reception unit 120, a processing unit 130, a storage unit 140, and an input receiving unit 150. The ultrasonic probe 200 includes an ultrasonic transducer device 210 and a force sensor 220. The ultrasonic image device 400 includes an ultrasonic measuring device 100 and a display unit 410. Note that the ultrasonic measuring device 100 and the ultrasonic image device 400 of this embodiment are not limited to those having the configurations shown in FIG. 1, and various modifications can be carried out, such as omitting some of the constituent elements, replacing some of the constituent elements with other constituent elements, and adding other constituent elements.

The ultrasonic transducer device 210 includes ultrasonic transducer elements. The ultrasonic transducer elements convert an emission signal, which is an electrical signal, into ultrasound, and convert an ultrasonic echo from a target subject (test subject) into an electrical signal. The ultrasonic transducer elements may be, for example, thin-film piezoelectric ultrasonic transducer elements, bulk piezoelectric ultrasonic transducer elements, capacitive micromachined ultrasonic transducer (CMUT) elements, or the like.

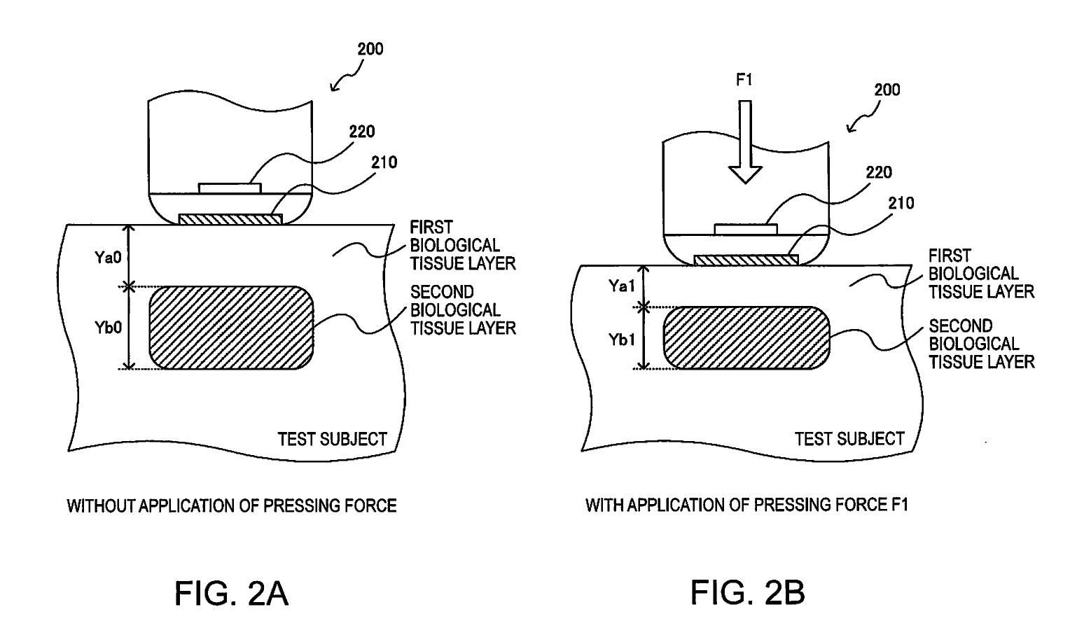

The force sensor 220 measures pressing force applied to the test subject, and outputs the measured pressing force to the processing unit 130. The pressing force refers to force applied to the test subject by the user pressing the ultrasonic probe 200 against the test subject. For example, as shown in FIGS. 2A and 2B, which will be described later, the force sensor 220 is provided on the opposite side of the ultrasonic transducer device 210 in the ultrasound emission direction. This prevents the force sensor 220 from affecting the emission and reception of ultrasound by the ultrasonic transducer device 210.

The emission unit 110 performs processing for emitting an ultrasonic beam. Specifically, the emission unit 110 generates and amplifies a pulse signal under control of the processing unit 130, and outputs an emission signal (drive signal), which is an electrical signal, to the ultrasonic transducer device 210. The ultrasonic transducer device 210 converts the emission signal, which is an electrical signal, into ultrasound and emits the ultrasound. The emission unit 110 may be configured by, for example, a pulse generator, an amplifier, or the like. It is also possible to provide at least a part of the emission unit 110 in the ultrasonic probe 200.

The reception unit 120 performs processing for receiving an ultrasonic echo obtained as a result of an ultrasonic beam being reflected by the test subject. Specifically, the ultrasonic transducer device 210 converts an ultrasonic echo from the target subject into an electrical signal, and outputs the electrical signal to the reception unit 120. The reception unit 120 performs reception processing, such as amplification, detection, A/D conversion and phase alignment, on a reception signal (analog signal), which is the electrical signal transmitted from the ultrasonic transducer device 210, and outputs the reception signal (digital data), which is a signal obtained as a result of the reception processing, to the processing unit 130. The reception unit 120 may be configured by, for example, a low noise amplifier, a voltage control attenuator, a programmable gain amplifier, a low-pass filter, an A/D converter, or the like. It is also possible to provide at least a part of the reception unit 120 in the ultrasonic probe 200.

The processing unit 130 performs processing for controlling the emission unit 110 and the reception unit 120, and processing for generating ultrasonic images based on reception signals from the reception unit 120. The processing unit 130 also obtains elasticity information of a biological tissue layer of the test subject based on thickness information of the biological tissue layer acquired from the reception signals from the reception unit 120 and pressing force information regarding the pressing force applied to the test subject from the force sensor 220. The method for obtaining the elasticity information will be described later in detail.

The thickness information of a biological tissue layer refers to information regarding the thickness (length in the depth direction) of the biological tissue layer, and encompasses not only the thickness value itself, but also an index that indicates the thickness. The pressing force information refers to information regarding the pressing force, and encompasses not only the pressing force value itself, but also an index that indicates the pressing force. The elasticity information of a biological tissue layer does not need to be the value of elastic constant (modulus of elasticity), and may be an index that indicates the hardness or softness of the biological tissue layer.

The processing unit 130 may be configured by, for example, a dedicated digital signal processor (DSP), or a general-purpose microprocessing unit (MPU). Alternatively, a part of processing executed by the processing unit 130 may be executed by a personal computer (PC).

The storage unit 140 includes, for example, a storage device such as, for example, a DRAM, and stores therein reception signals, ultrasonic image data and the like received from the processing unit 130, reads out the stored signals and data, and outputs them to the processing unit 130. The storage unit 140 may further include a nonvolatile storage device such as a flash memory so as to store elasticity information of biological tissue layers measured in the past. The processing unit 130 can detect a change in the elasticity information of the biological tissue layers over time by making comparison between the currently measured elasticity information and the elasticity information measured in the past and stored in the storage unit 140.

The input receiving unit 150 is an input device such as a keyboard or a touch panel, and receives commands, numerical values and the like input by the user, and outputs the received commands, numerical values and the like to the processing unit 130.

The display unit 410 is a display device such as, for example, a liquid crystal display, and receives display image data generated by the processing unit 130 and displays the display image data. The display image data includes, for example, an ultrasonic image (B mode image), the thickness information and elasticity information of the biological tissue layers, information that needs to be presented to the user, and the like.

With the ultrasonic measuring device 100 of this embodiment, the elasticity information of the biological tissue layers of the test subject can be obtained through ultrasonic measurement, and it is therefore possible to detect, for example, a lesion, such as lymphedema, having an elastic constant different from that of normal biological tissue layers, with high precision. Furthermore, the force sensor that measures pressing force does not affect the emission and reception of ultrasound, and thus the user can measure the elastic constant while viewing the ultrasonic image such as a B mode image during measurement. As a result, it is possible to efficiently detect a lesion.

2. Measurement of Elasticity Information

FIGS. 2A and 2B are diagrams illustrating measurement of elasticity information performed by the ultrasonic measuring device 100 of this embodiment. FIG. 2A shows an example of measurement without application of pressing force. FIG. 2B shows an example of measurement with application of pressing force F1. The test subject is composed of a first biological tissue layer and a second biological tissue layer having different elastic constants.

As shown in FIG. 2A, in the case where pressing force is not applied (initial state), the thickness (initial thickness information) of the first biological tissue layer is Ya0, and the thickness (initial thickness information) of the second biological tissue layer is Yb0. These thicknesses can be acquired based on an ultrasonic image obtained in the initial state (initial ultrasonic image).

As shown in FIG. 2B, in the case where first pressing force F1 is applied, the thickness of the first biological tissue layer changes to Ya1, and the thickness of second biological tissue layer changes to Yb1. These thicknesses can be acquired based on an ultrasonic image (first ultrasonic image) obtained in the state in which the first pressing force F1 is applied.

The following equation is given, where the elastic constant of the first biological tissue layer is represented by Ka, and the elastic constant of the second biological tissue layer is represented by Kb:

F1=Ka×ΔYa1+Kb×ΔYb1 (1),

where ΔYa1=Ya1−Ya0, and ΔYb1=Yb1−Yb0.

Also, although not shown, thicknesses Ya2 and Yb2 of the first and second biological tissue layers in the case where second pressing force F2 that is different from the first pressing force F1 is applied can be acquired based on an ultrasonic image (second ultrasonic image) obtained in the state in which the second pressing force F2 is applied. In this case as well, the following equation is given as with Equation (1):

F2=Ka×ΔYa2+Kb×ΔYb2 (2),

where ΔYa2=Ya2−Ya0, and ΔYb2=Yb2−Yb0.

The first and second pressing forces F1 and F2 can be detected by the force sensor 220, and thus the elastic constants Ka and Kb of the first and second biological tissue layers can be obtained from Equations (1) and (2).

It is also possible to obtain the elastic constants Ka and Kb of the first and second biological tissue layers by applying mutually different first to n-th (where n is an integer of 3 or greater) pressing forces F1 to Fn, and acquiring thickness variations ΔYa1 to ΔYan and ΔYb1 to ΔYbn from the ultrasonic images. By doing so, the elastic constants Ka and Kb can be obtained with higher precision.

As described above, with the ultrasonic measuring device 100 of this embodiment, ultrasonic measurement is performed by applying different first and second pressing forces, and thus elasticity information of the first and second biological tissue layers having different elastic constants can be obtained. By doing so, for example, a lesion such as lymphedema in a normal biological tissue layer can be detected with high precision. Furthermore, progression and the like of the lesion can be precisely recognized from the changes over time in the elasticity information of the lesion.

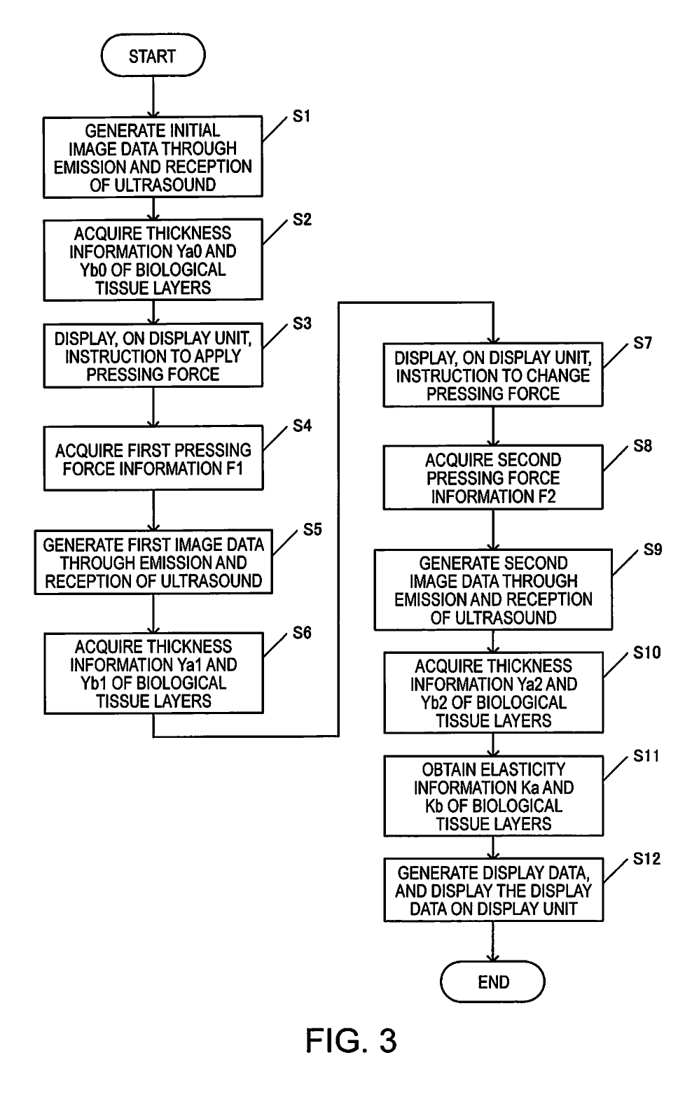

FIG. 3 shows an example of a flowchart of elasticity information measurement performed by the ultrasonic measuring device 100 of this embodiment. The processing shown in FIG. 3 is executed by the processing unit 130.

First, the processing unit 130 performs processing for emitting and receiving ultrasound in a state in which pressing force is not applied, so as to generate initial ultrasonic image data (B mode image data) based on a reception signal from the reception unit 120 (step S1). Processing for displaying, on the display unit 410, an instruction that prompts the user to take measurement without applying pressing force may be performed before the processing unit 130 performs the emission and reception processing.

Next, the processing unit 130 performs processing for analyzing the initial ultrasonic image data so as to acquire thickness information Ya0 and Yb0 of the first and second biological tissue layers in the initial state (step S2). To be specific, the processing unit 130 detects a boundary between the biological tissue layers by detecting luminance value peaks in the initial ultrasonic image data, and obtains thickness information Ya0 and Yb0 of the biological tissue layers from coordinate values in the depth direction of the detected boundary. As used herein, the coordinate values in the depth direction refers to coordinate values in the depth direction (the direction in which ultrasonic beams are emitted) of a single frame image of the B mode image, for example.

Next, the processing unit 130 performs processing for displaying, on the display unit 410, an instruction (message) that prompts the user to apply pressing force (step S3). Alternatively, the processing unit 130 may perform processing for providing an audio message that prompts the user to apply pressing force. Subsequently, the processing unit 130 acquires first pressing force information F1 based on detection information from the force sensor 220 (step S4).

Next, the processing unit 130 performs processing for emitting and receiving ultrasound in a state in which first pressing force F1 is applied, so as to generate first ultrasonic image data (step S5). Then, the processing unit 130 performs processing for analyzing the first ultrasonic image data so as to acquire thickness information (first thickness information) Ya1 and Yb1 of the first and second biological tissue layers in the state in which the first pressing force F1 is applied (step S6).

Next, the processing unit 130 performs processing for displaying, on the display unit 410, an instruction (message) that prompts the user to change the pressing force (step S7). Alternatively, the processing unit 130 may provide an audio message that prompts the user to change the pressing force. Subsequently, the processing unit 130 acquires second pressing force information F2 based on detection information from the force sensor 220 (step S8).

Next, the processing unit 130 performs processing for emitting and receiving ultrasound in a state in which second pressing force F2 is applied, so as to generate second ultrasonic image data (step S9). Then, the processing unit 130 performs processing for analyzing the second ultrasonic image data so as to acquire thickness information (second thickness information) Ya2 and Yb2 of the first and second biological tissue layers in the state in which the second pressing force F2 is applied (step S10).

Next, the processing unit 130 obtains elasticity information Ka and Kb of the first and second biological tissue layers based on the first and second pressing forces F1 and F2, and thickness variations ΔYa1, ΔYa2, ΔYb1 and ΔYb2 of the first and second biological tissue layers (step S11). Then, the processing unit 130 performs processing for generating display image data that includes the determined elasticity information Ka and Kb, an ultrasonic image and the like, and displaying the display image data on the display unit 410 (step S12).

The processing for analyzing ultrasonic image data does not necessarily need to be performed to acquire the thickness information of the biological tissue layers. The thickness information may be acquired by, for example, the user determining a boundary between biological tissue layers while viewing the ultrasonic image displayed on the display unit 410 and inputting the position of the boundary on the screen by moving a cursor or the like, and then the processing unit 130 determining the thickness information from coordinate values indicating the position of the boundary that has been input.

If the boundary between biological tissue layers is clearly seen in the ultrasonic image, the boundary between biological tissue layers can be detected by detecting luminance value peaks. However, if the boundary between biological tissue layers is not clearly seen, or if the boundary line is not linear but complex, it is difficult to obtain accurate thickness information from luminance value peaks.

With the ultrasonic measuring device 100 of this embodiment, feature points are extracted from ultrasonic images, and thus the thickness variations in the biological tissue layers can be acquired from the amounts of displacement of the feature points. Hereinafter, the method therefor will be described.

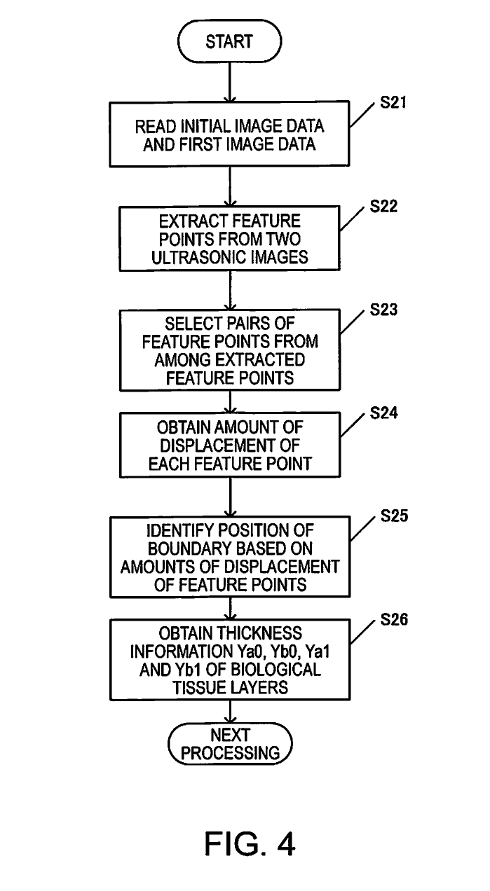

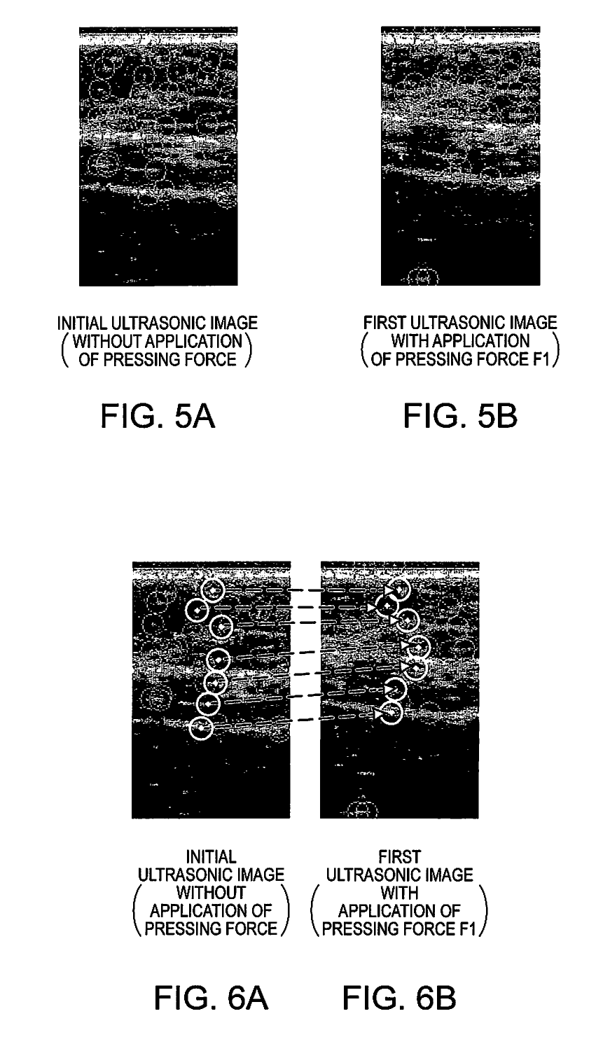

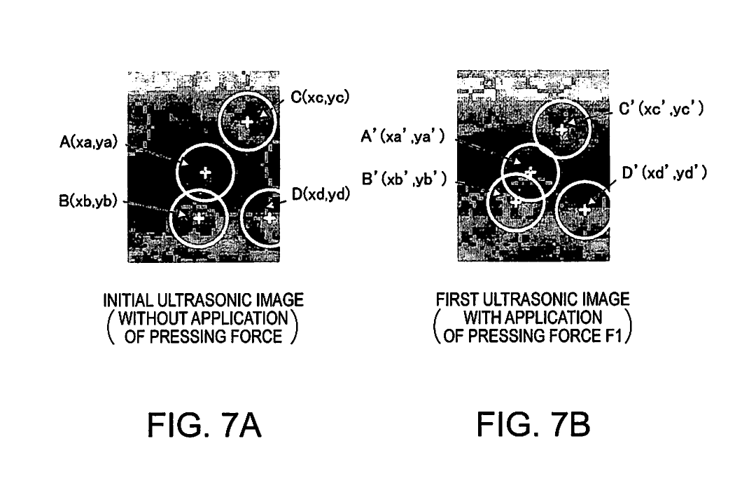

FIG. 4 shows an example of a flowchart of processing for acquiring the thicknesses of biological tissue layers through feature point extraction performed on ultrasonic images, performed by the ultrasonic measuring device 100 of this embodiment. The processing shown in FIG. 4 is executed by the processing unit 130. FIGS. 5A and 5B show an example of feature point extraction. FIGS. 6A and 6B show an example of feature point matching, and FIGS. 7A and 7B show an example of coordinate values of feature points.

As shown in FIG. 4, the processing unit 130 first reads, from the storage unit 140, initial ultrasonic image data and first ultrasonic image data (step S21). The initial ultrasonic image data refers to B mode image data obtained without application of pressing force, and the first ultrasonic image data refers to B mode image data obtained with application of first pressing force F1. In the following description, the initial ultrasonic image data will be referred to as “ultrasonic image A”, and the first ultrasonic image data will be referred to as “ultrasonic image B”.

Next, the processing unit 130 extracts feature points from the ultrasonic image A and the ultrasonic image B (step S22). The feature points refer to points that can be clearly observed in the images. In FIG. 5A, circles indicate feature points in the B mode image obtained without application of pressing force. In FIG. 5B, circles indicate feature points in the B mode image obtained with application of first pressing force F1.

In this example, a corner detection method is used as a method for extracting feature points, but other corner portion detection (eigenvalue or FAST feature detection) methods may be used. Alternatively, a local feature descriptor as typified by scale invariant feature transform (SIFT), a speeded up robust feature (SURF), or the like may be used.

Then, the processing unit 130 selects pairs of corresponding feature points from the group of feature points extracted in the two ultrasonic images (step S23). In other words, a feature point indicating the same portion as that in the ultrasonic image A is identified (estimated) from the group of feature points in the ultrasonic image B, and the two feature points are matched as a pair. In this example, the relationship between corresponding points is identified by using random sample consensus (RANSAC), but it is also possible to use other methods such as the least squares method, the least median method, and the M-estimation method.

As a specific example, results obtained by feature point matching performed on the ultrasonic image A and the ultrasonic image B are shown in FIGS. 6A and 6B. In the ultrasonic image A shown in FIG. 6A and the ultrasonic image B shown in FIG. 6B, each pair of matching feature points, which are indicated by white circles, is connected by an arrow. As shown in FIGS. 6A and 6B, not all feature points are matched. Also, not all feature points need to be matched. However, the precision of thickness variations that need to be obtained increases as the number of pairs of matching feature points increases.

Next, the processing unit 130 calculates the amount of displacement (displacement amount information) for each pair of matching feature points (step S24). The amount of displacement of a pair of feature points refers to the amount of movement that indicates how much a feature point has moved between two ultrasonic images. In this example, on the two-dimensional plane of the B mode image, the average value of variations in the distance between a feature point and other feature points that are in proximity to the feature point is obtained as a variation Δd of the feature point.

For example, as shown in FIGS. 7A and 7B, there are three feature points that are in proximity to a feature point A, namely, feature points B, C and D. A feature point A′ is the feature point corresponding to the feature point A, and the coordinate values of the feature point A are (xa, ya). The same applies to other feature points. In this case, the variation Δd of the feature point A is given by the following equation:

Δd=(D1+D2+D3)/3 (3),

where D1 represents a variation in the distance between the feature point A and the feature point B, D1=(distance between feature points A′ and B′)−(distance between feature points A and B), D2 represents a variation in the distance between the feature point A and the feature point C, and D3 represents a variation in the distance between the feature point A and the feature point D.

Next, the processing unit 130 identifies the boundary position based on the amount of displacement of each feature point (step S25). In the case where the first and second biological tissue layers have different elastic constants Ka and Kb, the amount of displacement at a feature point when pressing force is applied varies. For example, in the case of Ka>Kb, the amount of displacement at a feature point belonging to the second biological tissue layer is larger than that at a feature point belonging to the first biological tissue layer. This is because the second biological tissue layer deforms more significantly. Accordingly, by checking the distribution of the amount of displacement of feature points, it is possible to determine which of the first and second biological tissue layers each feature point belongs to. As a result, the boundary position between the first and second biological tissue layers can be identified.

Next, the processing unit 130 obtains thickness information of each biological tissue layer from the coordinate values of the identified boundary position between the biological tissue layers (step S26). According to this method, the position of the boundary between the biological tissue layers is identified in both the initial ultrasonic image and the first ultrasonic image, and it is therefore possible to obtain thickness information Ya0 and Yb0 in the initial state and thickness information Ya1 and Yb1 in the state in which the first pressing force F1 is applied.

In the same manner, the processing unit 130 can obtain thickness information Ya2 and Yb2 in a state in which second pressing force F2 is applied by extracting feature points from the initial ultrasonic image and a second ultrasonic image.

A part or a large part of processing of the ultrasonic measuring device 100 and the ultrasonic image device 400 of this embodiment may be implemented by a program. In this case, the ultrasonic measuring device 100 and the ultrasonic image device 400 of this embodiment are implemented by a processor such as CPU executing the program. To be specific, the program stored in an information storage medium is read out, and the readout program is executed by a processor such as CPU. The information storage medium (computer-readable medium) is a medium in which programs, data and the like are stored, and its function can be implemented by an optical disk (DVD, CD or the like), a HDD (hard disk drive), a memory (card-type memory, ROM or the like) or the like. The processor such as CPU performs various processing operations of this embodiment based on the program (data) stored in the information storage medium. That is, a program that causes a computer (an apparatus that includes an operation unit, a processing unit, a storage unit and an output unit) to function as respective units of this embodiment (a program for causing a computer to execute processing of respective units) is stored in the information storage medium.

3. Ultrasonic Transducer Element

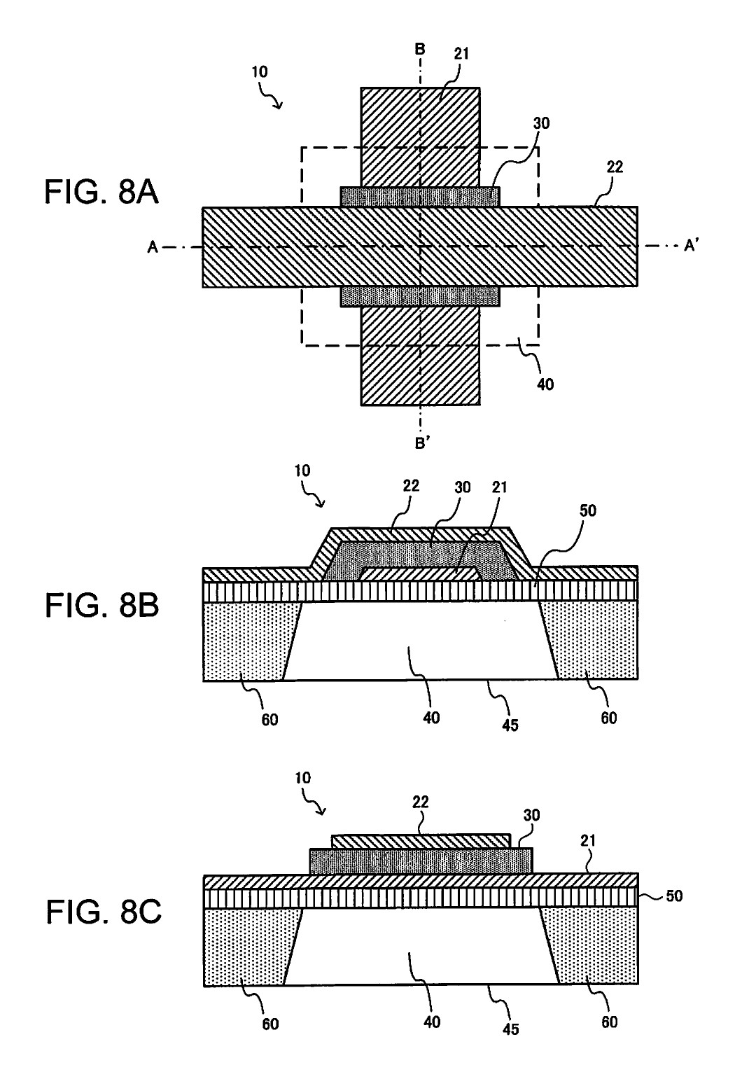

FIGS. 8A, 8B and 8C show a configuration example of an ultrasonic transducer element 10 included in the ultrasonic transducer device 210. The ultrasonic transducer element 10 includes a vibration film (membrane, support member) 50 and a piezoelectric element unit. The piezoelectric element unit includes a first electrode layer (lower electrode) 21, a piezoelectric layer (piezoelectric film) 30, and a second electrode layer (upper electrode) 22. Note that the ultrasonic transducer element 10 of this embodiment is not limited to that having the configuration shown in FIGS. 8A, 8B and 8C, and various modifications can be carried out, such as omitting some of the constituent elements, replacing some of the constituent elements with other constituent elements, and adding other constituent elements.

FIG. 8A is a plan view of the ultrasonic transducer element 10 formed on a substrate (silicon substrate) 60, as viewed from a direction normal to the surface of the substrate 60 on which the element is formed. FIG. 8B is a cross-sectional view showing a cross section taken along the line A-A′ shown in FIG. 8A. FIG. 8C is a cross-sectional view showing a cross section taken along the line B-B′ shown in FIG. 8A.

The first electrode layer 21 is formed by a thin metal film on the vibration film 50. As shown in FIG. 8A, the first electrode layer 21 may be an interconnect that extends to outside the element forming region and is connected to an adjacent ultrasonic transducer element 10.

The piezoelectric layer 30 is formed by, for example, a PZT (lead zirconate titanate) thin film provided so as to cover at least a part of the first electrode layer 21. The material of the piezoelectric film 30 is not limited to PZT, and it is also possible to use, for example, lead titanate (PbTiO₃), lead zirconate (PbZrO₃), lead lanthanum titanate ((Pb, La)TiO₃), and the like.

The second electrode layer 22 is formed by, for example, a thin metal film provided so as to cover at least a part of the piezoelectric film 30. As shown in FIG. 8A, the second electrode layer 22 may be an interconnect that extends to outside the element forming region and is connected to an adjacent ultrasonic transducer element 10.

The vibration film (membrane) 50 has a two-layer structure of, for example, a SiO₂ thin film and a ZrO₂ thin film, and is provided so as to close a cavity region 40. The vibration film 50 supports the piezoelectric film 30 and the first and second electrode layers 21 and 22, and is capable of generating ultrasound by being vibrated by extension and contraction of the piezoelectric film 30.

The cavity region 40 is formed by etching the back surface (the surface on which the element is not formed) of the substrate 60 (silicon substrate) by reactive ion etching (RIE) or the like. The resonance frequency of ultrasound is determined by the size of the vibration film 50 allowed to vibrate as a result of the cavity region 40 being formed, and the ultrasound is radiated toward the piezoelectric film 30 (from the rear side toward the front in FIG. 8A).

The lower electrode (first electrode) of the ultrasonic transducer element 10 is formed by the first electrode layer 21, and the upper electrode (second electrode) is formed by the second electrode layer 22. To be specific, a portion of the first electrode layer 21 that is covered by the piezoelectric film 30 serves as the lower electrode, and a portion of the second electrode layer 22 that covers the piezoelectric film 30 serves as the upper electrode. That is, the piezoelectric film 30 is sandwiched between the lower electrode and the upper electrode.

The piezoelectric film 30 extends and contracts in an in-plane direction by application of voltage between the lower electrode and the upper electrode, or in other words, between the first electrode layer 21 and the second electrode layer 22. The ultrasonic transducer element 10 has a monomorph (unimorph) structure in which a thin piezoelectric element unit and a vibration film 50 are bonded together, and thus when the piezoelectric element unit extends and contracts within the plane, the vibration film 50 bonded thereto is curved because the size of the vibration film 50 does not change. Accordingly, application of an alternating current voltage to the piezoelectric film 30 causes the vibration film 50 to vibrate in a thickness direction thereof, thereby ultrasound is radiated by the vibrations of the vibration film 50. The voltage applied to the piezoelectric film 30 is, for example, 10 to 30 V, and the frequency is, for example, 1 to 10 MHz.

A bulk ultrasonic transducer element has a peak-to-peak driving voltage of approximately 100 V, but the peak-to-peak driving voltage of the thin-film piezoelectric ultrasonic transducer element as shown in FIGS. 8A, 8B and 8C can be reduced to approximately 10 to 30 V.

4. Ultrasonic Transducer Device

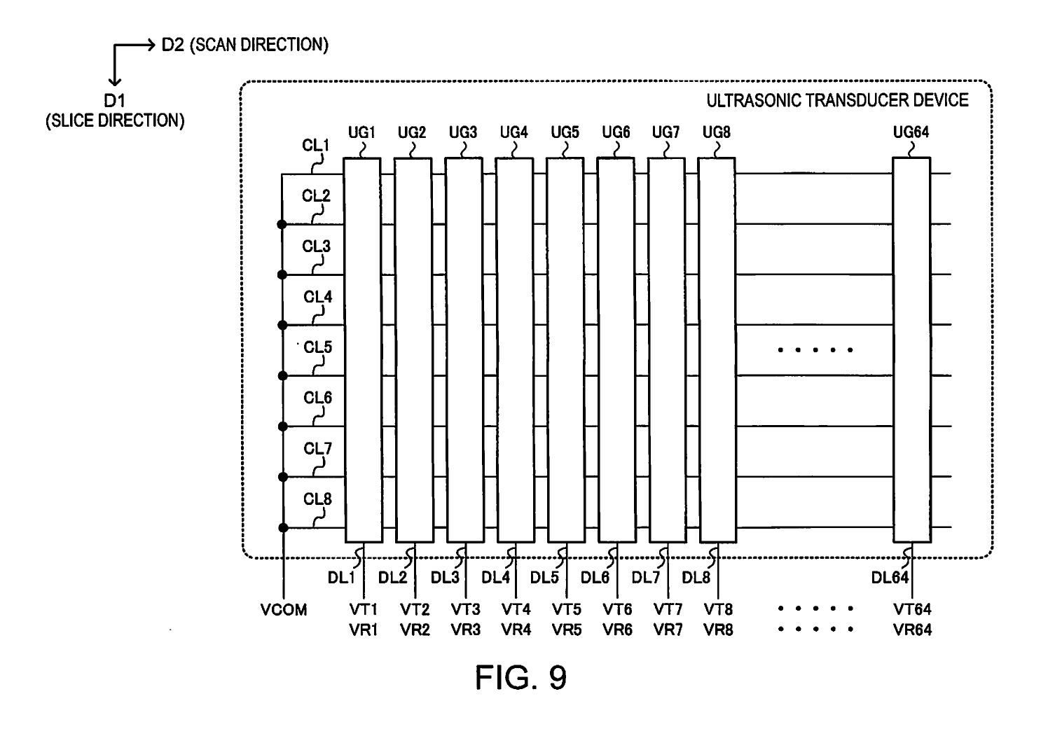

FIG. 9 shows a configuration example of the ultrasonic transducer device 210. The ultrasonic transducer device 210 of this configuration example includes a plurality of ultrasonic transducer element groups UG1 to UG64, driving electrode lines DL1 to DL64 (first to n-th driving electrode lines in a broad sense, n is an integer of 2 or greater), common electrode lines CL1 to CL8 (first to m-th common electrode lines in a broad sense, m is an integer of 2 or greater). The number (n) of driving electrode lines and the number (m) of common electrode lines are not limited to the numbers shown in FIG. 9.

The plurality of ultrasonic transducer element groups UG1 to UG64 are arranged along a second direction D2 (scan direction) in 64 columns. Each of the ultrasonic transducer element groups UG1 to UG64 includes a plurality of ultrasonic transducer elements that are arranged along a first direction D1 (slice direction).

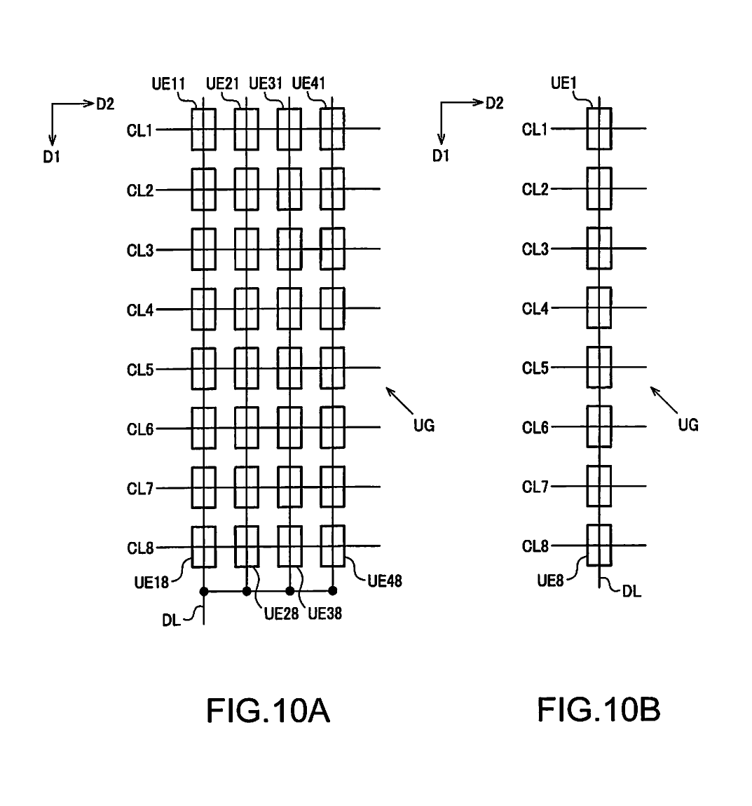

FIG. 10A shows an example of an ultrasonic transducer element group UG (UG1 to UG64). In FIG. 10A, the ultrasonic transducer element group UG is configured by first to fourth element columns. The first element column is configured by ultrasonic transducer elements UE11 to UE18 that are arranged along the first direction D1, and the second element column is configured by ultrasonic transducer elements UE21 to UE28 that are arranged along the first direction D1. The third element column (UE31 to UE38) and the fourth element column (UE41 to UE48) also have the same configuration. A driving electrode line DL (DL1 to DL64) is commonly connected to the first to fourth element columns. Also, common electrode lines CL1 to CL8 are connected to the ultrasonic transducer elements included in the first to fourth element columns.

The ultrasonic transducer element group UG shown in FIG. 10A constitutes one channel of the ultrasonic transducer device. That is, the driving electrode line DL corresponds to one channel's worth of driving electrode line, and one channel's worth of emission signal from an emission circuit is input into the driving electrode line DL. Also, one channel's worth of reception signal from the driving electrode line DL is output from the driving electrode line DL. The number of element columns that constitute one channel is not limited to four as shown in FIG. 10A, and may be less than or greater than four. For example, as shown in FIG. 10B, the number of element columns may be one.

As shown in FIG. 9, the driving electrode lines DL1 to DL64 (first to n-th driving electrode lines) are provided along the first direction D1. Among the driving electrode lines DL1 to DL64, a j-th (where j is an integer that satisfies 1≦j≦n) driving electrode line DLj (j-th channel) is connected to the first electrodes (for example, lower electrodes) of the ultrasonic transducer elements included in the j-th ultrasonic transducer element group UGj.

During an emission period in which ultrasound is radiated, emission signals VT1 to VT64 are supplied to the ultrasonic transducer elements via the driving electrode lines DL1 to DL64. During a reception period in which ultrasonic echo signals are received, reception signals VR1 to VR64 from the ultrasonic transducer elements are output via the driving electrode lines DL1 to DL64.

The common electrode lines CL1 to CL8 (first to m-th common electrode lines) are provided along the second direction D2. The second electrodes of ultrasonic transducer elements are connected to any one of the common electrode lines CL1 to CL8. Specifically, for example, as shown in FIG. 9, among the common electrode lines CL1 to CL8, an i-th (where i is an integer that satisfies 1≦i≦m) common electrode line CLi is connected to the second electrodes (for example, upper electrodes) of the ultrasonic transducer elements included in the i-th row.

A common voltage VCOM is supplied to the common electrode lines CL1 to CL8. It is sufficient that the common voltage VCOM is a constant direct current voltage, and it does not necessarily need to be 0 V, or in other words, the ground potential (earth potential).

During the emission period, a voltage, which is the difference between the emission signal voltage and the common voltage, is applied to the ultrasonic transducer elements, and ultrasound of a specific frequency is radiated.

The arrangement of the ultrasonic transducer elements is not limited to the matrix arrangement shown in FIG. 9. It is also possible to use a so-called staggered arrangement, or the like.

Also, FIGS. 10A and 10B show an example in which one ultrasonic transducer element is used as both an emitting element and a receiving element, but the embodiment is not limited thereto. It is possible to, for example, separately provide ultrasonic transducer elements for use as emitting elements and ultrasonic transducer elements for use as receiving elements, and arrange them in an array.

5. Ultrasonic Image Device

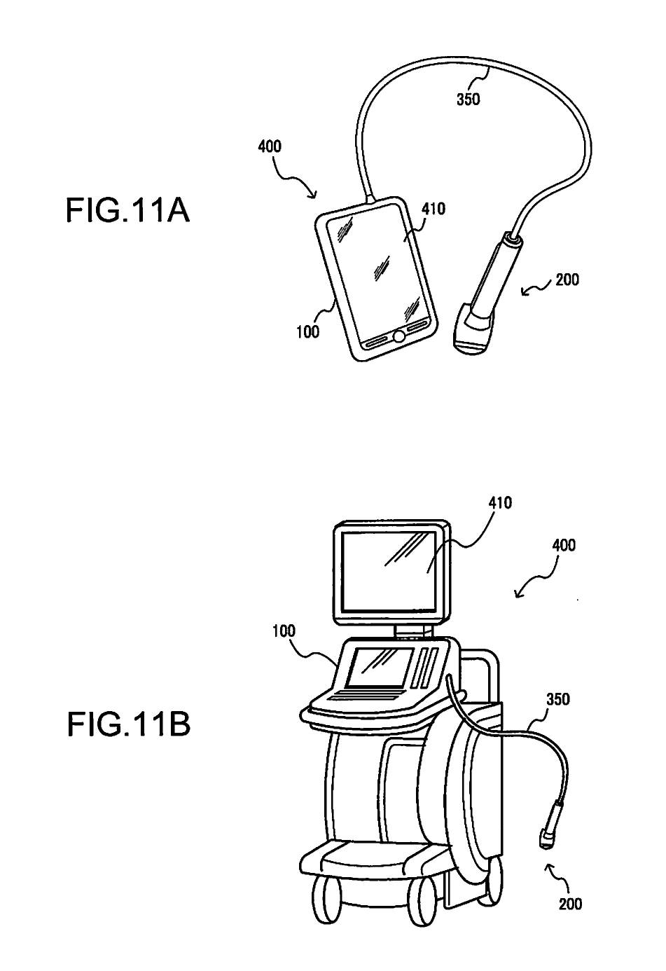

FIGS. 11A and 11B show specific configuration examples of the ultrasonic image device 400 of this embodiment. FIG. 11A shows a portable ultrasonic image device 400, and FIG. 11B shows a stationary ultrasonic image device 400.

The portable and stationary ultrasonic image devices 400 both include the ultrasonic measuring device 100, the ultrasonic probe 200, a cable 350, and the display unit 410. The ultrasonic probe 200 includes the ultrasonic transducer device 210, and is connected to the ultrasonic measuring device 100 via the cable 350. The display unit 410 displays display image data.

At least a part of the emission unit 110, the reception unit 120, and the processing unit 130 of the ultrasonic measuring device 100 may be provided in the ultrasonic probe 200.

Note that although embodiments have been explained in detail above, a person skilled in the art will readily appreciate that it is possible to implement numerous variations and modifications that do not depart substantially from the novel aspects and effect of the invention. Accordingly, all such variations and modifications are also to be included within the scope of the invention. For example, terms that are used within the description or drawings at least once together with broader terms or alternative synonymous terms can be replaced by those other terms at other locations as well within the description or drawings. Also, the configuration and operation of the ultrasonic measuring device and the ultrasonic image device, and the ultrasonic measuring method are not limited to those described in the embodiments, and various modifications are possible.

The entire disclosure of Japanese Patent Application No. 2013-145225, filed Jul. 11, 2013 is expressly incorporated by reference herein.

What is claimed is:

1. An ultrasonic measuring device comprising:
an ultrasonic transducer device;
a force sensor that measures pressing force;
an emission unit that performs processing for emitting an ultrasonic beam;
a reception unit that performs processing for receiving an ultrasonic echo obtained from the ultrasonic beam being reflected by a test subject; and
a processing unit that performs analysis processing based on a reception signal from the reception unit and detection information from the force sensor,
wherein the processing unit obtains elasticity information of a biological tissue layer of the test subject based on thickness information and pressing force information, the thickness information being thickness information of the biological tissue layer acquired based on the reception signal from the reception unit, and the pressing force information being pressing force information regarding the pressing force applied to the test subject from the force sensor.

2. The ultrasonic measuring device according to claim 1,
wherein if the biological tissue layer includes a first biological tissue layer and a second biological tissue layer that have different elastic constants,
the processing unit acquires first pressing force information as the pressing force information, and first thickness information of the first biological tissue layer and first thickness information of the second biological tissue layer as the thickness information when the pressing force applied to the test subject during measurement is a first pressing force, acquires second pressing force information as the pressing force information, and second thickness information of the first biological tissue layer and second thickness information of the second biological tissue layer as the thickness information when the pressing force applied to the test subject during measurement is a second pressing force that is different from the first pressing force, and obtains elasticity information of the first biological tissue layer and elasticity information of the second biological tissue layer based on the first pressing force information, the second pressing force information, the first thickness information of the first biological tissue layer, the first thickness information of the second biological tissue layer, the second thickness information of the first biological tissue layer, and the second thickness information of the second biological tissue layer that have been acquired.

3. The ultrasonic measuring device according to claim 2,
wherein where the first pressing force is represented by F1, the second pressing force is represented by F2, the first thickness of the first biological tissue layer is represented by Ya1, the first thickness of the second biological tissue layer is represented by Yb1, the second thickness of the first biological tissue layer is represented by Ya2, the second thickness of the second biological tissue layer is represented by Yb2, initial thickness of the first biological tissue layer is represented by Ya0, initial thickness of the second biological tissue layer is represented by Yb0, the elasticity of the first biological tissue layer is represented by Ka, and the elasticity of the second biological tissue layer is represented by Kb, the processing unit obtains the elasticity Ka of the first biological tissue layer and the elasticity Kb of the second biological tissue layer from the following relational equations:
F1=Ka×(Ya1−Ya0)+Kb×(Yb1−Yb0); and
F2=Ka×(Ya2−Ya0)+Kb×(Yb2−Yb0).

4. The ultrasonic measuring device according to claim 1,
wherein the processing unit generates ultrasonic image data based on a reception signal from the reception unit, analyzes the ultrasonic image data so as to acquire thickness information of the biological tissue layer, and obtains the elasticity information of the biological tissue layer based on the acquired thickness information of the biological tissue layer and the pressing force information.

5. The ultrasonic measuring device according to claim 4,
wherein the processing unit detects a boundary in the biological tissue layer by detecting a luminance value peak in the ultrasonic image data, and obtains the thickness information of the biological tissue layer from coordinate values in a depth direction of the detected boundary in the biological tissue layer.

6. The ultrasonic measuring device according to claim 4,
wherein the processing unit extracts a feature point of the biological tissue layer in the ultrasonic image data, and detects the boundary in the biological tissue layer based on displacement amount information of the feature point by application of the pressing force on the test subject.

7. An ultrasonic image device comprising:
the ultrasonic measuring device according to claim 1; and
a display unit that displays display image data generated by the processing unit.

8. An ultrasonic image device comprising:
the ultrasonic measuring device according to claim 2; and
a display unit that displays display image data generated by the processing unit.

9. An ultrasonic image device comprising:
the ultrasonic measuring device according to claim 3; and
a display unit that displays display image data generated by the processing unit.

10. An ultrasonic image device comprising:
the ultrasonic measuring device according to claim 4; and
a display unit that displays display image data generated by the processing unit.

11. An ultrasonic image device comprising:
the ultrasonic measuring device according to claim 5; and
a display unit that displays display image data generated by the processing unit.

12. An ultrasonic image device comprising:
the ultrasonic measuring device according to claim 6; and
a display unit that displays display image data generated by the processing unit.

13. An ultrasonic measuring method comprising:
performing processing for emitting an ultrasonic beam to a test subject;
performing processing for receiving an ultrasonic echo obtained from the ultrasonic beam being reflected by the test subject;
acquiring pressing force information regarding pressing force applied to the test subject during measurement; and
obtaining elasticity information of a biological tissue layer of the test subject based on the pressing force information and thickness information of the biological tissue layer based on a reception signal of the ultrasonic echo.
